(19) United States
(12) Patent Application Publication
(10) Pub. No.: US 2026/0263126 A1
(43) Pub. Date: Sep. 10, 2026

(54) Bone Plate for Toe Valgus Correction

(71) Applicant: A Plus Biotechnology Company Limited, New Taipei City (TW)

(72) Inventors: Chia-Hung Chu, Taichung city (TW); Ya-Han Chan, Pingtung County (TW); Tsung-An Hsieh, New Taipei City (TW); Shih-Chieh Wang, Taichung City (TW)

(21) Appl. No.: 19/537,893

(22) Filed: Feb. 12, 2026

(30) Foreign Application Priority Data
Mar. 6, 2025 (TW) 114108392
Apr. 14, 2025 (TW) 114114040

(51) Int. Cl. A61B 17/80; A61B 17/86

(52) U.S. Cl. CPC A61B 17/8061; A61B 17/8057; A61B 17/809; A61B 17/86

(57) ABSTRACT

The present invention discloses a bone plate for toe valgus correction, which includes a long plate body having a distal part, a curved part and a proximal tip part along its long axis. The first locking hole, the second locking hole and the third locking hole, are respectively disposed on the surface of the distal part, the curved part. A suture hole is provided on the side of the curved portion. The distal end portion and the proximal tip portion are located on different planes, and are connected by the curved portion.

### TECHNICAL FIELD

The present invention relates to medical device, more particularly, to a bone plate for correcting hallux valgus.

### BACKGROUND

Hallux valgus is a common condition where the big toe excessively tilts outwards towards the side of a foot. It typically refers to the big toe deviating more than 15 degrees towards the second toe, causing the big toe to overlap the second toe and resulting in misplacement. This usually causes discomfort from friction when wearing shoes, and when barefoot, the bone at the base of the big toe may protrude, and it causes redness, swelling, and pain may easily occur.

While ill-fitting shoes play a role in the formation of hallux valgus, the main cause is a defect in the bones that make up the foot. Secondly, prolonged standing and walking can also increase ligament damage, which can promote the onset of the disease or accelerate its progression. Furthermore, frequently wearing pointed shoes or high heels can cause problems. The triangular shape of the pointed shoes forces the front of the foot into a narrow triangular area. Combined with the lack of elasticity of the upper leather of the shoes, this can cause the big toe to turn outwards and the little toe to turn inwards. If there is significant compression of the second toe by the big toe, it should be corrected promptly. Once the symptoms have become quite severe and the pain is unbearable, affecting daily life, surgery may be considered to alleviate the pain and discomfort, in order to achieve bone reconstruction and soft tissue balance.

Bunionectomy is quite diverse, mainly consisting of "soft tissue repair" and "osteotomy correction". The former relaxes the tight adductor muscles on the outside, and tightens the loose joint capsule and ligaments on the inside; the latter corrects the outward-turned big toe to the ideal angle.

When treating hallux valgus surgically, the first metatarsal bone that is turned inward must be corrected to the ideal angle through osteotomy to achieve the desired effect. Currently, there are many surgical methods for correcting hallux valgus. Each osteotomy method has its own advantages and disadvantages. In addition, the internal fixation devices used, such as steel nails and screws, may have problems such as low stability or insufficient corrective force.

Currently, the bone screws and plates used in the surgical treatment of hallux valgus have some shortcomings in use. For example, referring to the conventional bone plate 10 for treating hallux valgus shown in FIG. 4A, it is implanted into the medullary cavity through a cut incision in the first metatarsal 30. The distal end of the bone plate 10 is fixed by only one bone screw 210, which may cause the distal osteotomy of the first metatarsal 30 to rotate relative to the proximal osteotomy after surgery, thus affecting postoperative healing.

In order to address the above-mentioned deficiencies, it is necessary to provide new types of hallux valgus correction implants by the present invention, such as novel spear-shaped locking plates, which can provide patients with better options.

### SUMMARY OF THE INVENTION

To achieve the above objective, the present invention discloses a bone plate for correcting hallux valgus, comprising: an elongated plate body, which sequentially includes a distal end, a curved portion, and a proximal tip along its long axis; the first locking hole and the second locking hole are provided on the surface of the distal end; the third locking hole is provided on the surface of the curved portion; and a suture hole is provided on the side of the curved portion. The distal end and the proximal tip are located on different planes and are connected by the curved portion.

In one embodiment, the distal end, the curved portion, and the proximal tip are integrally formed to create a spear-shaped bone plate. The suture hole is located on the side of the bone plate between the second locking hole and the third locking hole, and extends from one side to the other, providing suture for the joint capsule of the big toe to correct hallux valgus (HVA).

In one embodiment, the inner walls of the first locking hole, the second locking hole, and the third locking hole have internal threads, and they are all threaded holes that penetrate the upper and lower surfaces, providing a gripping force for locking the bone screw. The outer side of the bone screw has continuous external threads that correspond to the internal threads of the inner walls of the first locking hole, the second locking hole, and the third locking hole, providing an engagement between the external threads and the internal threads.

In one embodiment, first included angle is formed between the second locking hole and the first locking hole, the angle of which ranges from 0° to 15°. The third locking hole is an angled locking hole, and the second included angle is formed between the third locking hole and the first locking hole, the angle of which ranges from 0° to 60°.

In one embodiment, the step for the bone plate to correct hallux valgus includes to insert the proximal tip of the bone plate through an incision cut into the medullary cavity of the first metatarsal bone of the big toe for fixation. The bone screws are inserted through the first locking hole, the second locking hole, and the third locking hole into the cut first metatarsal bone for fixation, such that the bone plate is securely connected to the cut first metatarsal bone by the curved portion and proximal tip, ensuring correct alignment to correct the intermetatarsal angle (IMA) between the first and second metatarsals.

In one embodiment, the outer edge of the bone plate is designed with a low-profile to conform to the curvature of the medullary cavity wall of the first metatarsal, thereby increasing the fit when implanted into the medullary cavity.

### BRIEF DESCRIPTION OF THE DRAWINGS

FIG. 1A shows a perspective view of a bone plate for correcting hallux valgus disclosed in the present invention.

FIG. 1B shows a top view of a bone plate for correcting hallux valgus according to the present invention.

FIG. 1C shows a side view of a bone plate for correcting hallux valgus according to the present invention.

FIG. 2 shows a cross-sectional view of a bone plate for correcting hallux valgus disclosed in the present invention in the AA direction of FIG. 1B.

FIG. 3 shows a top view of a bone plate disclosed in the present invention for correcting hallux valgus after osteotomy, implanted and fixed.

FIG. 4A shows a side view of a conventional treatment for hallux valgus, where a corrective bone plate is implanted and fixed after osteotomy.

FIG. 4B shows a side view of a bone plate for correcting hallux valgus disclosed in the present invention after osteotomy and implantation and fixation.

### DETAILED DESCRIPTION

The present invention is described in detail with reference to specific embodiments and concepts. It is understood that these descriptions are for illustrative purposes only to explain the structure or operation of the invention, and are not intended to limit the scope of the claims of the present invention. Accordingly, the invention is not limited to the specific and preferred embodiments set forth herein but is capable of being embodied in various other forms. The implementation of the present invention is described hereinafter by way of specific embodiments. Those skilled in the art will readily appreciate the effectiveness and advantages of the present invention through the content disclosed in this specification. It is further understood that the present invention may be applied and implemented through other specific embodiments, and the details set forth in this specification may be modified or varied based on different requirements without departing from the spirit of the present invention.

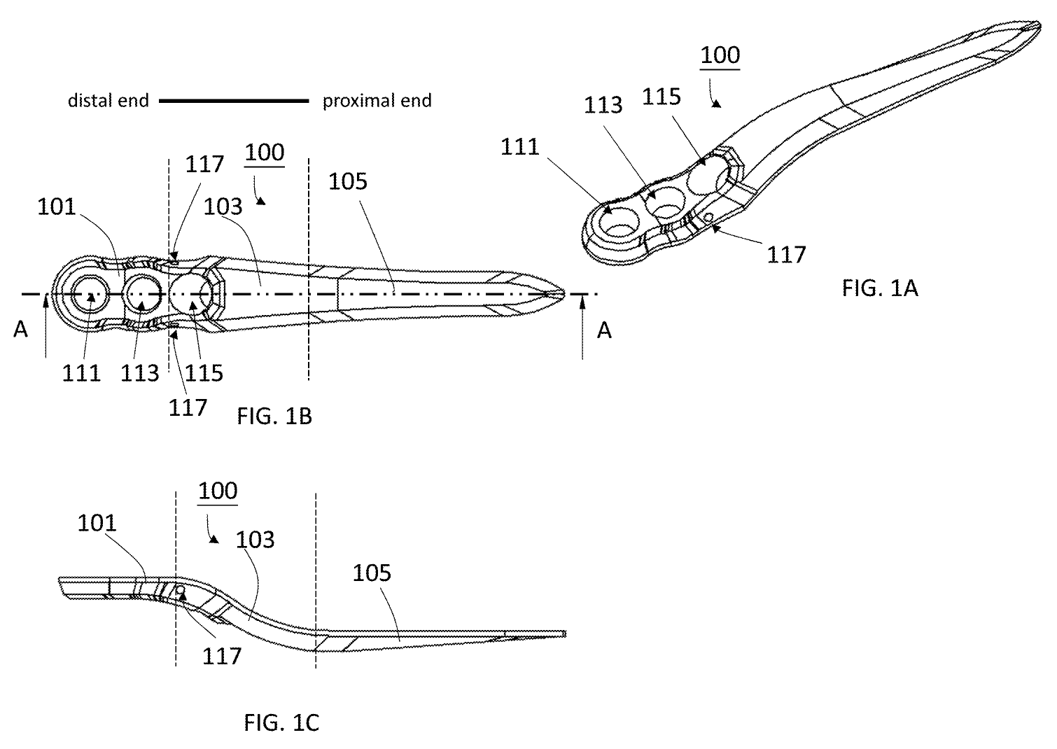

The present invention mainly discloses a bone plate 100 for correcting hallux valgus. Please refer to FIGS. 1A-1C, which respectively show a perspective view, a top view, and a side view of the bone plate 100. The bone plate 100 is an elongated plate body, which, along its long axis, sequentially includes a distal end 101, a curved portion 103, and a proximal tip 105 from the distal end to the proximal end; wherein the distal end 101 and the proximal tip 105 are located in different planes and are connected by the curved portion 103. The distal end 101, the curved portion 103, and the proximal tip 105 are integrally formed to form a spear-shaped bone plate. Along its long axis, the plate has the first locking hole 111 and the second locking hole 113 on the distal end surface, the third locking hole 115 on the curved portion surface, and a suture hole 117 on the side of the curved part.

According to an embodiment of the present invention, the inner walls of the first locking hole 111, the second locking hole 113 and the third locking hole 115 have internal threads, and they are all threaded holes that penetrate the upper and lower surfaces, providing a gripping force for locking the bone screw; the suture hole 117 is provided on the side between the second locking hole 113 and the third locking hole 115, and passes through from one side to the other side, providing suturing for the joint capsule of the big toe to correct the hallux valgus angle (HVA).

Referring to FIGS. 1A and 1B, the outer edge of the bone plate 100 is designed with a low-profile, which can better conform to the curvature of the medullary cavity wall of the big toe, especially, it can increase the fit when it is implanted into the medullary cavity of the first metatarsal bone.

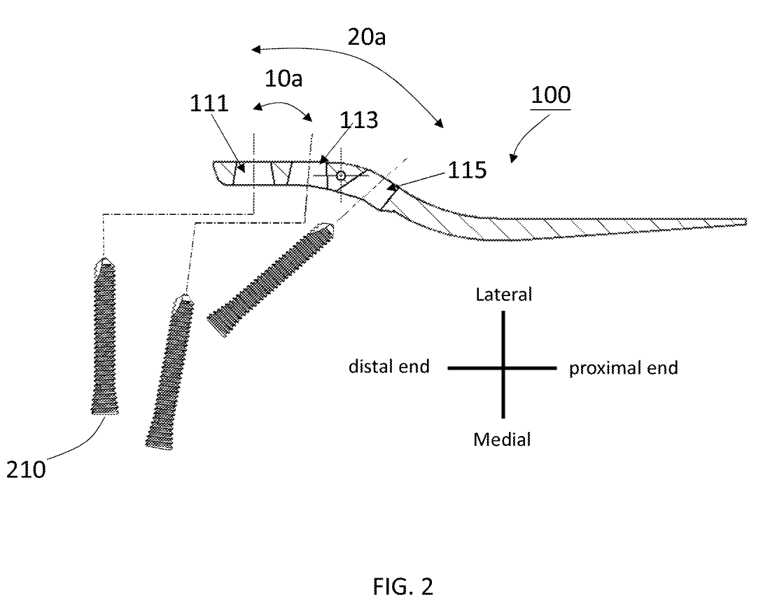

FIG. 2 shows a cross-sectional view of the bone plate 100 along the AA direction (refer to FIG. 1B). Each bone screw 210 can be respectively inserted into the first locking hole 111, the second locking hole 113 provided on the distal end surface, and the third locking hole 115 provided on the curved portion surface. According to an embodiment of the present invention, the outer wall of the bone screw 210 has a continuous external thread that corresponds to the internal thread of the inner wall of the first locking hole 111, the second locking hole 113, and the third locking hole 115, so that the external thread can be engaged with the internal thread of the inner wall of the first locking hole 111, the second locking hole 113, and the third locking hole 115. According to an embodiment of the present invention, the first locking hole 111 is perpendicular to the distal end surface. A small angle, the first included angle 10 a, is formed between the second locking hole 113 and the first locking hole 111, the angle of which ranges from 0° to 15°. The third locking hole 115 is an oblique locking hole, and the second included angle 20a is formed between the third locking hole 115 and the first locking hole 111, the angle of which ranges from 0° to 60°.

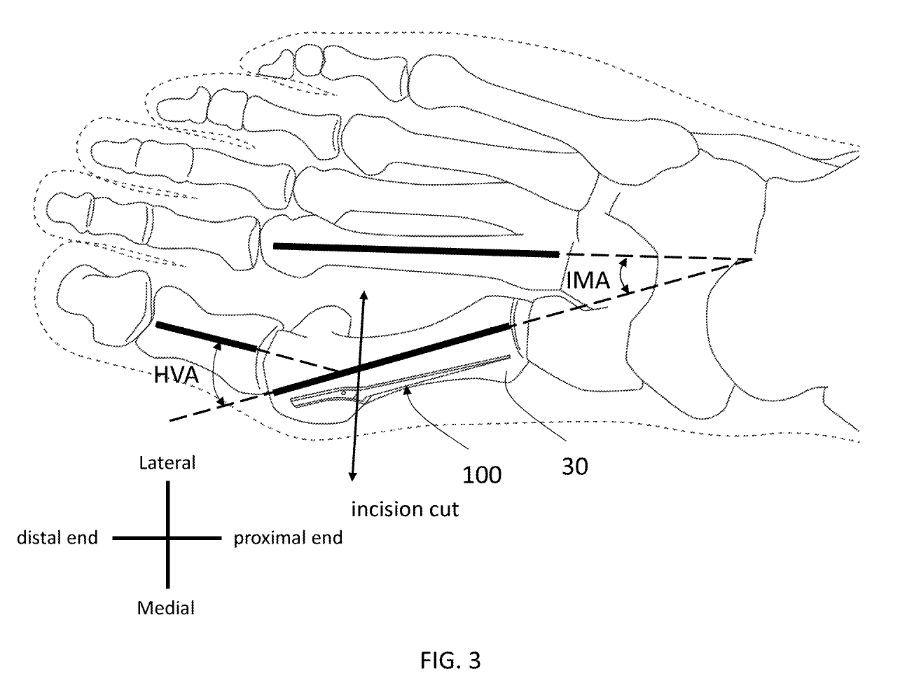

Referring to FIG. 3, when treating hallux valgus surgically, the first metatarsal 30 is first cut along the incision direction. The proximal tip 105 of the bone plate 100 is inserted into the medullary cavity of the first metatarsal 30 through the cut surface for fixation. Bone screws are then inserted into the cut first metatarsal 30 through the first locking hole 111 (refer to FIG. 1), the second locking hole 113 (refer to FIG. 1), and the third locking hole 115 (refer to FIG. 1) for fixation. This allows the bone plate to firmly connect the cut first metatarsals to each other through the curved portion and the proximal tip, ensuring correct alignment to correct the intermetatarsal angle (IMA) between the first and second metatarsals. In addition, with the assistance of the suture hole 117, sutures can be sutured the joint capsule to correct the hallux valgus angle (HVA).

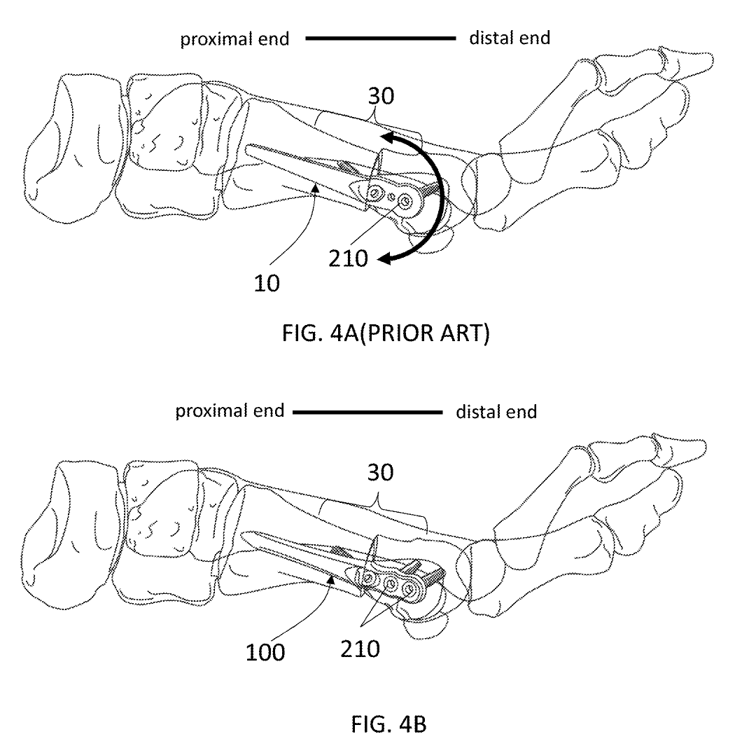

Compared with the conventional art in FIG. 4A, the bone plate 100 disclosed in this invention, referring to FIG. 4B, has a design that provides two screws 210 for locking at its distal end, which can effectively prevent the distal osteotomy of the first metatarsal 30 from rotating relative to the proximal osteotomy after fixation. Therefore, the present invention can avoid the defects such as slow postoperative healing and insufficient stability that may be caused by the bone plate 10 of the conventional art (FIG. 4A).

The above invention is a corrective implant specifically developed for hallux valgus surgery—a spear-shaped next-generation locking bone plate, which patients can choose from. Its advantages include: (1) minimally invasive incision, reduced soft tissue damage, good blood circulation, and rapid wound recovery; (2) depending on the situation, different angles of bone plates can be selected to make various degrees of correction, with high corrective force; (3) short operation time; (4) twice the stability of other implants; (5) conforms well without protrusion and without foreign body sensation; (6) applicable to various correction methods. Three months after the surgery, the recovery can be tracked via X-ray. Once the bones have healed, the patient can start exercising, such as swimming, cycling, running, and jumping.

The above description details the preferred embodiments of the present invention. It will be appreciated by those skilled in the art that these embodiments are provided for purposes of illustration and are not intended to limit the scope of patent protection sought for the invention. The scope of the invention is defined by the claims appended hereto and any equivalents thereof. Any changes, alterations, or modifications made by those skilled in the art without departing from the spirit or scope of the claims shall be considered equivalent variations or designs accomplished within the spirit of the present disclosure and are intended to be included within the scope of the following claims.

What is claimed is:

1. A bone plate for correcting hallux valgus, comprising:
an elongated plate body sequentially including a distal end, a curved portion, and a proximal tip along a long axis;
wherein said distal end has a first locking hole and a second locking hole provided on said distal end’s surface, said curved portion having a third locking hole provided on said curved portion’s surface, and said curved portion having a suture hole provided on said curved portion’s side; and
wherein said distal end and said proximal tip are located in different planes and are connected by said curved portion.

2. The bone plate of claim 1, wherein said distal end, said curved portion, and said proximal tip are integrally formed to create a spear-shaped bone plate.

3. The bone plate of claim 1, wherein said suture hole is located on side of said bone plate between said second locking hole and said third locking hole, and extending from one side to another side, providing a suture through a joint capsule of said big toe to correct a hallux valgus angle (HVA).

4. The bone plate of claim 1, wherein said first locking hole, said second locking hole, and said third locking hole are threaded holes that penetrate from an upper surface to a lower surface, thereby providing a gripping force for bone screws.

5. The bone plate of claim 4, wherein said bone screws are engaged with said first locking hole, said second locking hole and said third locking hole.

6. The bone plate of claim 4, wherein a first included angle is formed between said second locking hole and said first locking hole, said first included angle being 0°~15°.

7. The bone plate of claim 4, wherein said third locking hole is an oblique locking hole, and a second included angle being formed between said third locking hole and said first locking hole, said second included angle being 0° to 60°.

8. The bone plate of claim 4, further comprising fixing said bone plate by inserting said proximal tip of said bone plate through a cut surface into an internal medullary cavity of said first metatarsal bone of said big toe.

9. The bone plate of claim 8, further comprising inserting said bone screws into said first metatarsal bone being cut for fixation, so that said bone plate being engaged to said first metatarsal bone by said curved portion and said proximal tip.

10. The bone plate of claim 9, wherein an outer edge of said bone plate features a low-profile design to conform to a curvature of said medullary cavity wall of said first metatarsal, thereby increasing fit when said bone plate is implanted into said medullary cavity.
